(19) United States
(12) Patent Application Publication
(10) Pub. No.: US 2020/0000397 A1
(43) Pub. Date: Jan. 2, 2020

(54) METHOD AND DEVICE FOR DETERMINING A LEVEL OF COLONISATION OF ACNE BACTERIA ON SKIN

(71) Applicant: Henkel AG & Co. KGaA, Duesseldorf (DE)

(72) Inventors: ANDREAS BOCK, Neuss (DE); THOMAS WELSS, Duesseldorf (DE); CLAUDIA HUNDEIKER, Meerbusch (DE); MARIANNE WALDMANN-LAUE, Monheim (DE)

(73) Assignee: Henkel AG & Co. KGaA, Duesseldorf (DE)

(21) Appl. No.: 16/481,578

(22) PCT Filed: Mar. 1, 2018
(86) PCT No.: PCT/EP2018/055024

§ 371 Date: Jul. 29, 2019

(30) Foreign Application Priority Data
Mar. 16, 2017 (DE) 10 2017 204 365.2

(51) Int. Cl. A61B 5/00; C07D 403/14

(52) U.S. Cl. CPC A61B 5/445; A61B 5/0071; A61B 5/0077; C07D 403/14; A61B 5/486; A61B 5/0013; A61B 5/0022

(57) ABSTRACT

In various exemplary embodiments, a method for determining a degree of colonization of skin with acne bacteria is provided. The method may comprise: for at least one region of a user's skin, recording an image of the region of skin by a camera during illumination of the region of skin with ultraviolet and/or blue light, wherein the camera is configured to at least capture light in a fluorescence wavelength range of a fluorescent agent produced by the acne bacteria, by the image, determining a captured quantity of light which is emitted from the fluorescent agent as fluorescence as a consequence of the illumination, and allocating a degree of colonization of the skin to the determined captured quantity of light, wherein the allocation of the degree of colonization of the skin to the determined captured quantity of light is carried out by a database stored in cloud computing architecture.

### TECHNICAL FIELD

This application is a U.S. National-Stage entry under 35 U.S.C. 35 U.S.C. § 371 based on International Application No. PCT/EP2018/055024, filed Mar. 1, 2018, which was published under PCT Article 21(2) and which claims priority to German Application No. 10 2017 204 365.2, filed Mar. 16, 2017, which are all hereby incorporated in their entirety by reference.

### BACKGROUND

In general, various embodiments relate to a method and a device for determining a degree of colonization of skin with acne bacteria (also described by the abbreviation “degree of colonization”, “degree of colonization of skin” or “degree of colonization with bacteria”) and a method for identifying a cosmetic skin treatment recommendation.

Bacteria which colonize the skin, for example the bacterium Propionibacterium acne, may be the cause of redness and inflammation in the skin. They may be responsible for the occurrence of blackheads (comdones) and spots, and even of acne.

A variety of specialized cosmetic products for the face and/or the body are available for the care of skin with acne, for example covering makeup and covering care products, inflammation-inhibiting and/or breathable decorative cosmetics, etc. However, the effectiveness of the cosmetic products may be strongly dependent on the individual physiology of the skin, for example on a degree of colonization of the skin with acne bacteria, which can be correlated with a degree to which acne may manifest itself.

Consumers want an even-looking complex without unsightly blemishes such as redness, blackheads or spots.

Without specialist dermatological or cosmetic advice, it can be difficult for a user to determine their individual skin condition and cosmetics which are suitable for their skin condition.

However, when the user uses cosmetic products, it could be almost impossible to achieve success with a treatment because the user, who is at home, for example, does not have the opportunity to assess the results of a treatment in a standardized, objective manner.

Thus, it may be made difficult for the consumer to assess an individual effectiveness of a cosmetic, which could lead to motivation for carrying out an appropriate cosmetic treatment, for example over a prolonged period, being compromised. This could even be the case when a cosmetic product would be suitable for obtaining an objectively demonstrable desired effect.

In many areas of daily life, for some time now there has been a trend towards personalized programs which can deal with individual requirements and needs, for example in a nutritional or health field, but also in the area of personalized cosmetics. This could enable a user to find specific cosmetic products and/or to obtain care tips which are tailored to the individual requirements of their skin, and thus lead to particularly high effectiveness.

### SUMMARY

In various exemplary embodiments, a device for determining a degree of colonization of skin with acne bacteria (hereinafter also occasionally abbreviated to degree of colonization or degree of colonization of skin). The determination of the degree of colonization of the skin and continuous monitoring of changes can, in various exemplary embodiments, offer the user an additional use of an individualizable cosmetic treatment.

In various exemplary embodiments, in order to determine the degree of colonization of the skin with acne bacteria, a sensor may be used which can quantify the number of bacteria on the skin. In this regard, quantification of the bacteria can be determined indirectly via the amount of endogenous porphyrin. The quantification may be carried out using the orange-red fluorescence of the endogenous porphyrin. To this end, excitation of porphyrin contained in the bacteria (for example Propionibacterium acne) by an illuminating device which emits light at a long wavelength UV-A and/or in the blue light range (for example what is known as a Wood lamp) may be necessary.

In various exemplary embodiments, the sensor may have a camera which may be configured to detect fluorescent light which is emitted from the excited porphyrin.

In various exemplary embodiments, the device for determining the degree of colonization of skin may comprise electronic circuitry, which may be configured to determine the captured quantity of light which is emitted from the fluorescent agent as fluorescence as a consequence of the illumination in the image, and to allocate a degree of colonization of the skin to the determined captured quantity of light by a database.

In various exemplary embodiments, a device may be provided which is connected to a smartphone, a tablet, a laptop or another computer device, or may be integrated into it.

In various exemplary embodiments, the device may exchange data with cloud computing architecture (also abbreviated to “the cloud”) by a data connection. As an example, using the cloud computing architecture, a database can be provided which provides an allocation of a respective quantity of light which is emitted by one of the fluorescent agents produced by the acne bacteria to a degree of colonization of the skin, wherein the database may have been determined as a reference database, for example in laboratory tests, and optionally may be updated by using measurement data from other users.

In various exemplary embodiments, by employing an app or other software, a degree of colonization of the skin with acne bacteria may be determined and provided in the form of a value (for example with arbitrary units), as a graphical representation and/or voice message.

In various exemplary embodiments, a degree of colonization of the skin with acne bacteria may be displayed over a longer period of time and monitoring/follow-up of the degree of colonization of the skin may be enabled by a (for example graphical) representation of the measurement results over time.

In various exemplary embodiments, the device may be inexpensive and small (for example portable) in configuration. The device may be used on a (living) user, for example by the user at home, at a point of sale of cosmetics, and/or in a beauty salon. In various exemplary embodiments, the device may be a smartphone, a tablet, an iPad or similar electronic circuitry (for example a data processing device and/or a data transmission device), and an integrated device or an add-on device which, for example, can be applied and/or connected to the electronic circuitry (for example connectable by a data connection). The integrated device or the additional device (optionally even the entire device) may be of a size which allows it to be accommodated easily in a handbag or trouser pocket, for example with an area of less than 36 cm² and with a thickness of less than 2 cm.

In various exemplary embodiments, fluorescence signals captured using the camera and the illuminating device may be compared with reference fluorescence signals for known degrees of bacterial colonization, and with the aid of these, a degree of bacterial colonization for the skin of the user may be determined (wherein the comparison may be carried out in a manner such that the signal is converted with the aid of a relationship to a degree of bacterial colonization of the user determined using reference signals and reference data). In various exemplary embodiments, the reference data may be determined in advance, for example during laboratory tests, and may be continuously updated by including data from other users, wherein the reference data may be provided in the form of a database.

In various exemplary embodiments, the comparison of the signal with the reference signal and/or the determination of the degree of bacterial colonization from it may be carried out by the electronic circuitry (for example directly), for example by an app or any other software program which is operated by the electronic circuitry (for example a smartphone, a tablet, an iPad or the like).

In various exemplary embodiments, the database may be provided by cloud computing architecture, for which the electronic circuitry may provide a (for example contactless) data communications connection.

In various exemplary embodiments, the database may be stored in the electronic circuitry.

In various exemplary embodiments, the comparison of the fluorescence signal with the reference fluorescence signal for determining the degree of bacterial colonization may be carried out directly by the electronic circuitry.

In various exemplary embodiments, the comparison of the fluorescence signal with the reference fluorescence signal for determining the degree of bacterial colonization may be carried out indirectly by the electronic circuitry, for example in that the electronic circuitry provides a (for example contactless) data communications connection and, by the data communications connection, the fluorescence signal is sent to an external data processing device (for example cloud computing architecture) and receives the degree of bacterial colonization of the skin determined by the fluorescence signal from the external data processing device.

In various exemplary embodiments, in order to determine the degree of bacterial colonization of the skin, a (physical) dimension of a region of the skin is determined for which a fluorescence is captured by the camera. In various exemplary embodiments, the dimension of the skin region, for example as described below, may be determined with the aid of the image (for example with the aid of structures in the image which have a defined typical dimension (unit of length or area)). In various exemplary embodiments, the dimension of the region of skin may be determined using a distance between the camera and the region of skin using properties of the camera (for example a magnification and a physical detector factor), for example as described below. In this manner, the distance between the camera and the region of skin may be predetermined and/or measured using a distance measuring device, for example as described below.

In various exemplary embodiments, the user may be provided with the determined degree of bacterial colonization of the skin in any manner, for example selectable by the user, for example as a numerical value, a graphical representation (for example a representation of the determined value as a ratio of the total range of values, from low colonization via moderate colonization to severely colonized), a voice message or the like.

Particularly in the case in which the degree of colonization is determined for a plurality of regions of skin, the provision of the degree of bacterial colonization of the skin to the user may comprise a graphical representation, for example a display, for example by a display device. The graphical representation may display the determined degree of bacterial colonization of the skin of the plurality of regions by encoding with the aid of the degree of bacterial colonization of the skin in a representation of the user (for example a diagrammatic representation or on a photograph of the user). As an example, the image of the user taken with the camera (for example a digital photograph which, for example, may show the user's face or a part of the user's face) may be used in order to show places with bacterial colonization, for example as a false colour representation, or with contours overlaid on the fluorescent intensity image.

In various exemplary embodiments, for example when a temporal profile of the degree of bacterial colonization is represented, different representations of the degree of bacterial colonization may be used for different points in time, for example different colours in the false colour representation, a false colour representation for one point in time and a different coloured contour for another point in time, or the like.

In various exemplary embodiments, the display device may be part of the device, for example a display on a smartphone, tablet or iPad. In various exemplary embodiments, the display device may be coupled to the device, for example by a data connection (for example cordless).

In various exemplary embodiments, the electronic circuitry may be configured to determine the degree of colonization itself, i.e. directly, for example using software, for example an app which may be installed on the electronic circuitry, wherein the software (for example the app) may be configured to use a database in order to determine the degree of colonization which may be provided by cloud computing architecture, or it may be cloud computing architecture.

In various exemplary embodiments, a method and a device for determining a degree of colonization of skin with acne bacteria may be provided. The device, the method and/or the degree of colonization determined by the device or the method may be used in order to determine the needs for the skin of the user.

In various exemplary embodiments, the user is empowered to find specific cosmetic products and/or to obtain care tips which can be tailored to individual requirements of the user's skin.

In order to determine the degree of bacterial colonization of the skin, in various exemplary embodiments, software, for example an app, may be used which, for example, may be installed on a portable data processing device (for example on a smartphone, a tablet, an iPad or the like). A camera which may be configured to capture a fluorescence signal of a fluorescent agent which is produced by acne bacteria, may be connected to the data processing device or be integrated into it. Furthermore, an illuminating device for ultraviolet and/or blue light may be provided, by which the skin of the user can be illuminated in order to produce the fluorescence signal. An assessment of the captured fluorescence signal can then enable the degree of colonization of the skin with acne bacteria to be quantified; in this regard, by a database which may be provided by the cloud, a degree of colonization of the skin with acne bacteria can be allocated to the fluorescence signal. The use of the database provided by the cloud means that even after providing the device to the user, the statistical reliability of the data in the database can be improved and optionally, the range of data regarding the degree of colonization that is covered can be broadened.

In various exemplary embodiments, by the portable data processing device, for example by the software/app, the determined degree of colonization may be provided or communicated as a value (for example with arbitrary units), as a verbal communication, as a graphical representation (for example as a visual representation which shows regions of the skin with acne bacteria colonization) or the like.

In various exemplary embodiments, at least one cosmetic product which corresponds to their individual requirements may be recommended to the user based on the determined degree of colonization. The cosmetic product may be suitable for maintaining the condition of the user's skin or for improving it (for example to reduce a degree of colonization with acne bacteria or at least not to raise it). In various exemplary embodiments, the cosmetic product may be a cream, a lotion, an ointment, an oil, an emulsion, a gel, a soap or the like, for example makeup, a powder, a blemish stick, a concealer or a covering care product such as a tinted day cream, for example.

In various exemplary embodiments, the product recommendations and/or care tip may be provided, for example, by software, for example an app. As an example, the user may be recommended to apply a cosmetic care product which has relatively high coverage to a region of skin which is relatively severely colonized with acne bacteria, and a cosmetic care product which has relatively low coverage to a region of skin which is not colonized or is only sparsely colonized with acne bacteria.

As an example, the user may be recommended to use or avoid products with specific ingredients. As an example, the user may be recommended to apply a cosmetic care product which contains benzoyl peroxide to a region of skin which is relatively severely colonized with acne bacteria. In addition to care products with benzoyl peroxide, the user may be recommended to use care products with antiseptics such as, for example, a combination of ammonia dodecylsulphate and dodecylbenzenesulphonic acid, sodium bituminosulphonate (shale oil), salicylic acid, retinoids or antibiotics. In contrast, the user may be discouraged from using care products with a high proportion of comedogenic ingredients.

In various exemplary embodiments, the software/app which determines the degree of bacterial colonization of the skin may be the same as that which determines the product recommendation and/or the care tip. In various exemplary embodiments, different software programs/apps may be used for a portion of the various procedures or for all of the various procedures (determination of degree of bacterial colonization of the skin, determination of a product recommendation, determination of care tip).

In various exemplary embodiments, the success of a treatment with a cosmetic treatment which may be aimed at reducing the determined degree of bacterial colonization of the skin with acne bacteria, may be monitored. In various exemplary embodiments, the software/app may enable a check and/or follow-up of the results by a representation (for example a graphical representation, for example as illustrated above) of the measurement results over time.

In various exemplary embodiments, when determining the product and care recommendations, further information may be employed regarding a general state of health, a skin condition, eating habits and other behaviour patterns of the user (for example time spent daily outdoors/in the sun/in water, smoking habits etc), for example by the software/app. In various exemplary embodiments, the software/app may request this information from the user.

In various exemplary embodiments, in order to assess a suitability of a care product and/or a care tip for caring for skin with a given skin condition, data from literature may be used as a basis therefor.

In various exemplary embodiments, a care product and/or a care tip may be assessed as suitable for a skin condition when it is to be expected that, for example, on the basis of data from the literature, experimental results or empirical values (which, for example, may be continuously updated, for example even when the software/app is being used by the user, with new empirical values from other users, see below), that during a (for example regular) use of the care product and/or the care tips, the degree of bacterial colonization of the user's skin is reduced, or at least not raised.

In various exemplary embodiments, an assessment of a suitability of a care product for improving the condition of the skin may be confirmed or modified by recording empirical values from other users with the same or a similar degree of colonization, for example from empirical values concerning successful treatment. This means that it could be possible for the user to always receive an optimized recommendation. It is advantageous if the other users whose empirical values are recorded also have a similar profile as regards age, gender, weight, general state of health and/or living habits in addition to the same or a similar degree of colonization.

In various exemplary embodiments, a check and follow-up of the effectiveness of a cosmetic treatment may be made possible in an objective and standardized manner. In various exemplary embodiments, the cosmetic treatment may be aimed at reducing a degree of bacterial colonization of the skin and/or be aimed at another cosmetic effect, for example a decorative effect, without raising the degree of bacterial colonization of the skin.

In various exemplary embodiments, the effectiveness of a (for example cosmetic) treatment can be followed up better, meaning that selection of an individually tailored product is or will be facilitated.

In various exemplary embodiments, a motivation of the user may be enhanced in order to carry out a cosmetic treatment over a longer term, for example by a comparison with other users, for example by information regarding successful treatment obtained from other users.

In various exemplary embodiments, a method for determining a degree of colonization of skin with acne bacteria is provided. The method may comprise: for at least one region of a user's skin, during illumination of the region of skin with ultraviolet and/or blue light, recording an image of the region of skin using a camera, wherein the camera is configured to at least capture light in a fluorescence wavelength range of a fluorescent agent produced by the acne bacteria, by the image, determining a captured quantity of light which is emitted from the fluorescent agent as fluorescence as a consequence of the illumination, and allocating a degree of colonization of the skin to the determined captured quantity of light, wherein the allocation of the degree of colonization of the skin to the determined captured quantity of light is carried out by a database stored in cloud computing architecture.

In various exemplary embodiments, the method may furthermore comprise updating the database based on new allocations of the degree of colonization of the skin to the determined captured quantity of light from a plurality of other users.

In various exemplary embodiments, the method may furthermore comprise recording a background light image of the region of skin using the camera while the region of skin is not illuminated with the ultraviolet and/or blue light, wherein the determination of the captured quantity of light which is emitted from the fluorescent agent as fluorescence as a consequence of the illumination, may comprise a determination of a difference between the image and the background light image.

In various exemplary embodiments, the fluorescent agent may comprise a porphyrin, preferably coproporphyrin III.

In various exemplary embodiments, the fluorescence wavelength range may be from about 560 nm to about 780 nm.

In various exemplary embodiments, the at least one region of skin comprises a plurality of regions of the user's skin.

In various exemplary embodiments, the camera and an illuminating device for producing the ultraviolet and/or blue light may be parts of at least one portable device.

In various exemplary embodiments, the portable device may comprise a smartphone, a tablet or an iPad or be capable of being attached to a smartphone, a tablet or an iPad.

In various exemplary embodiments, the determination of the captured quantity of light and/or the allocation of the degree of colonization of the skin to the determined captured quantity of light may be carried out by an app.

In various exemplary embodiments, a method for determining a cosmetic skin treatment recommendation is provided. The method may comprise a determination of a skin condition in accordance with various exemplary embodiments, and a determination of the cosmetic product and/or a care tip based on the determined degree of colonization of the skin and a further database stored in the cloud computing architecture which comprises a plurality of degrees of colonization and a plurality of associated cosmetic products and/or care tips, wherein each skin condition of the plurality of skin conditions may be associated with at least one suitable cosmetic product and/or at least one care tip.

In various exemplary embodiments, the cosmetic product and/or the care tips may be suitable for the degree of colonization of the skin when, based on empirical values stored in the further database from a plurality of other users of the cosmetic product, a reduction in the degree of colonization of the skin is to be expected.

In various exemplary embodiments, the determination of the degree of colonization of the skin and/or the determination of the cosmetic skin treatment recommendation may comprise transmitting the image and/or the degree of colonization of the skin to an external data processing device and receiving the degree of colonization of the skin and/or the cosmetic skin treatment recommendation.

In various exemplary embodiments, a device for determining a degree of colonization of skin with acne bacteria is provided. The device may comprise an illuminating device for illuminating at least one region of a user's skin with ultraviolet and/or blue light, a camera to record an image of the region of skin during the illumination of the region of skin by the illuminating device, wherein the camera is configured to at least capture light in a fluorescence wavelength range of a fluorescent agent produced by the acne bacteria, electronic circuitry which may be configured to determine, in the image, a captured quantity of light which is emitted from the fluorescent agent as fluorescence as a consequence of the illumination, and to allocate a degree of colonization of the skin to the determined captured quantity of light by a database; and a data exchange device for exchanging data between the electronic circuitry and cloud computing architecture, wherein by the data exchange device, the database stored in the cloud computing architecture can be made available for the allocation of the degree of colonization to the quantity of light.

In various exemplary embodiments, the fluorescence wavelength range may be from about 590 nm to about 760 nm.

In various exemplary embodiments, the ultraviolet and/or blue light may be in a wavelength range of between about 380 nm and about 500 nm.

In various exemplary embodiments, the ultraviolet and/or blue light may be in a wavelength range of between about 315 nm and about 380 nm.

In various exemplary embodiments, the device may furthermore comprise a filter the transmittance of which may be configured and which may be disposed in a manner such that the ultraviolet and/or blue light of the illuminating device is prevented from being captured by the camera, whereas the filter may be transparent to the light in the fluorescence wavelength range.

In various exemplary embodiments, the illuminating device, the camera and the electronic circuitry may be parts of at least one portable device.

In various exemplary embodiments, the camera and the electronic circuitry may be parts of an integrated portable device.

In various exemplary embodiments, the integrated portable device may comprise a smartphone, a tablet or an iPad.

In various exemplary embodiments, the data exchange device may comprise a data exchange device for contactless data exchange.

In various exemplary embodiments, a device for determining a degree of colonization of skin with acne bacteria is provided. The device may comprise an illuminating device for illuminating at least one region of a user's skin with ultraviolet and/or blue light, a camera to record an image of the region of skin during the illumination of the region of skin by the illuminating device, wherein the camera may be configured to at least capture light in a fluorescence wavelength range of a fluorescent agent produced by the acne bacteria, a distance measuring device for measuring a distance between the distance measuring device and the region of skin, and electronic circuitry which may be configured to determine, in the image, a captured quantity of light which is emitted from the fluorescent agent as fluorescence as a consequence of the illumination, to determine a dimension for the region of skin taking the camera properties into consideration, the measured distance between the distance measuring device and the region of skin and a distance between the distance measuring device and the camera, and to allocate a degree of colonization of the skin to the determined captured quantity of light by a database taking the dimension of the region of skin into consideration.

In various exemplary embodiments, the device may furthermore comprise a distance measuring device for measuring a distance between the distance measuring device and the region of skin, wherein the determination of the dimension of the region of skin may be based on the distance measuring device-region of skin distance, a camera-distance measuring device distance and camera properties. The camera properties may, for example, include a magnification and a physical dimension of a camera detector.

In various exemplary embodiments, a method for determining a degree of colonization of skin with acne bacteria is provided. The method may comprise, for at least one region of a user's skin, recording an image of the region of skin by a camera during illumination of the region of skin with ultraviolet and/or blue light, wherein the camera may be configured to capture at least light in a fluorescence wavelength range of a fluorescent agent produced by the acne bacteria, in the image, determining a captured quantity of light which is emitted from the fluorescent agent as fluorescence as a consequence of the illumination, and determining a dimension of the region of skin, and, by a database, allocating a degree of colonization of the skin to the determined captured quantity of light taking the dimension of the region of skin into account.

### BRIEF DESCRIPTION OF THE DRAWINGS

In the drawings, similar reference numerals usually refer to the same parts in all of the various views, wherein for the purposes of clarity, numbering all of the relevant parts in all of the figures is occasionally dispensed with. Parts themselves or the like may also be provided with a communal reference numeral with a following number or a following lower case letter. The drawings are not necessarily to the correct scale, because the emphasis is far more on illustrating the principles of the present disclosure. In the following description, various embodiments of the present disclosure are described with reference to the accompanying drawings, in which:

FIG. 1A and FIG. 1B respectively show a device for determining a degree of colonization of skin with acne bacteria in accordance with various exemplary embodiments and illustrate a use of the device;

FIG. 2 shows a device for determining a degree of colonization of skin with acne bacteria in accordance with various exemplary embodiments;

FIGS. 3A and 3B respectively show a device for determining a degree of colonization of skin with acne bacteria in accordance with various exemplary embodiments, wherein FIG. 3A furthermore illustrates a use of the device;

FIG. 4 illustrates a degree of colonization of skin with acne bacteria determined in accordance with various exemplary embodiments;

FIG. 5 shows a flow diagram to illustrate a method for determining a cosmetic product for skin treatment in accordance with various exemplary embodiments;

FIG. 6 shows a flow diagram for a method for determining a degree of colonization of skin with acne bacteria in accordance with various exemplary embodiments;

FIG. 7 shows a flow diagram for a method for determining a degree of colonization of skin with acne bacteria in accordance with various exemplary embodiments; and

FIG. 8 shows a flow diagram for a method for determining a cosmetic skin treatment recommendation in accordance with various exemplary embodiments.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The following comprehensive description refers to the accompanying drawings which show, by way of example by illustration, specific details and implementations regarding how the present disclosure can be put into practice.

The word “by way of example” as used herein has the meaning “as an example, an implementation or an illustration”. All embodiments or implementations described herein as “by way of example” should not be considered to necessarily be preferred or advantageous over other embodiments or implementations.

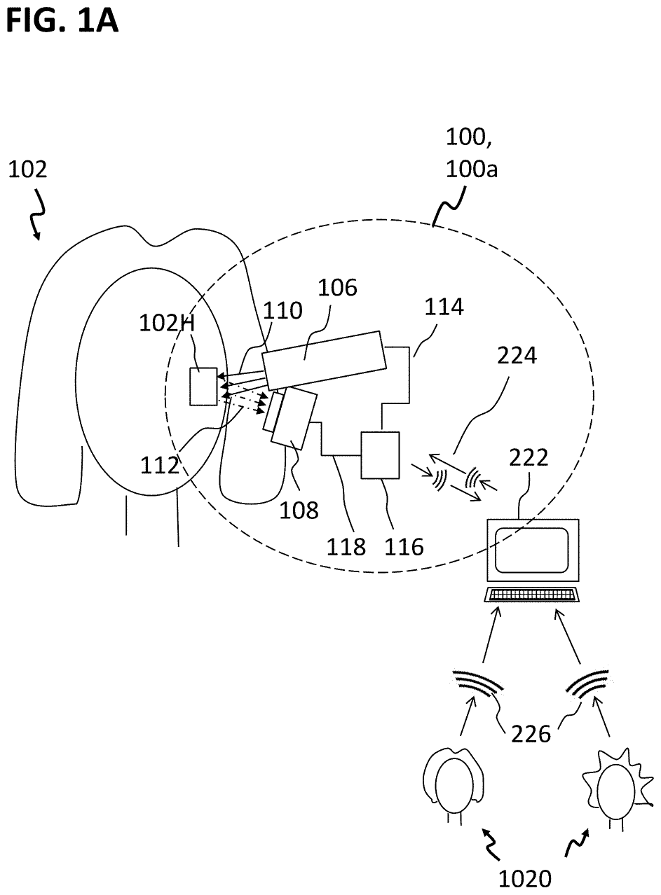

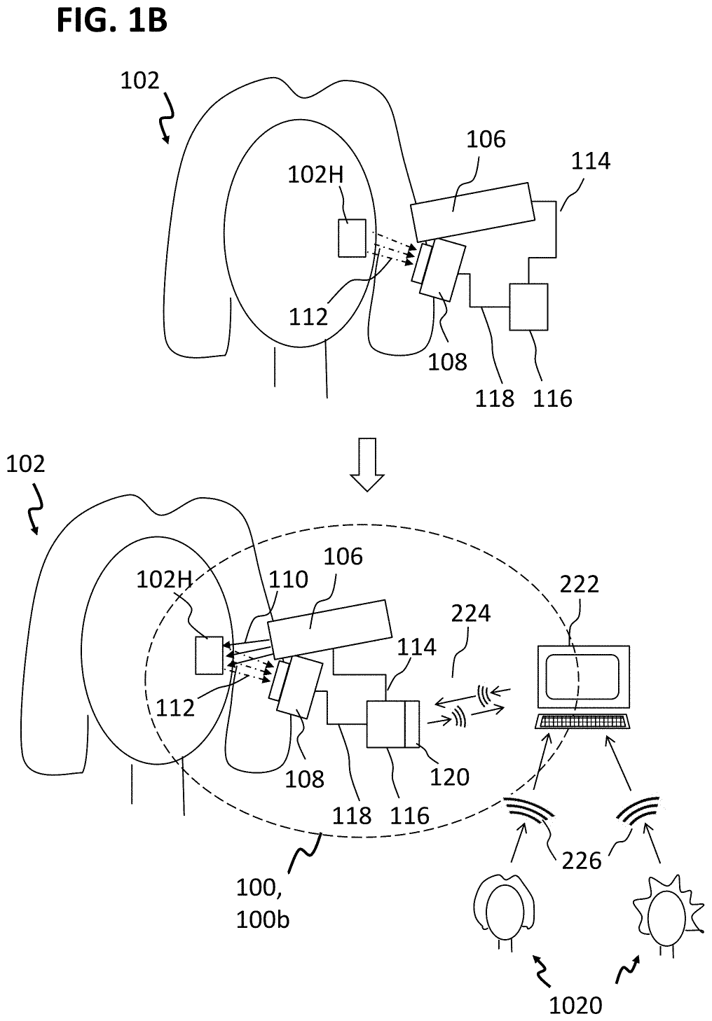

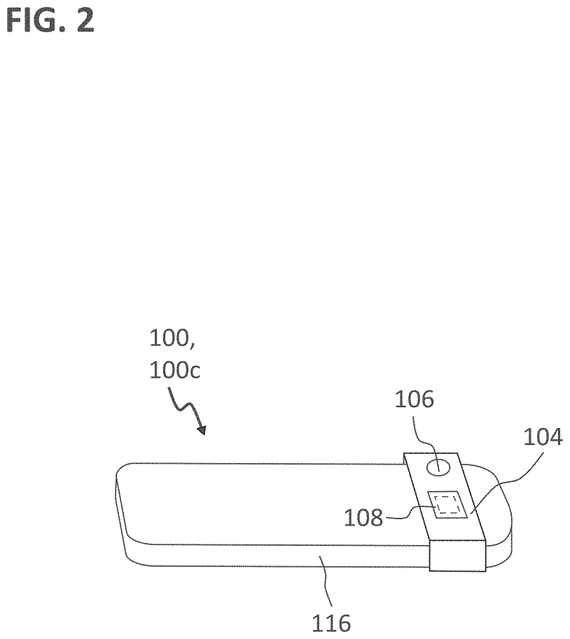

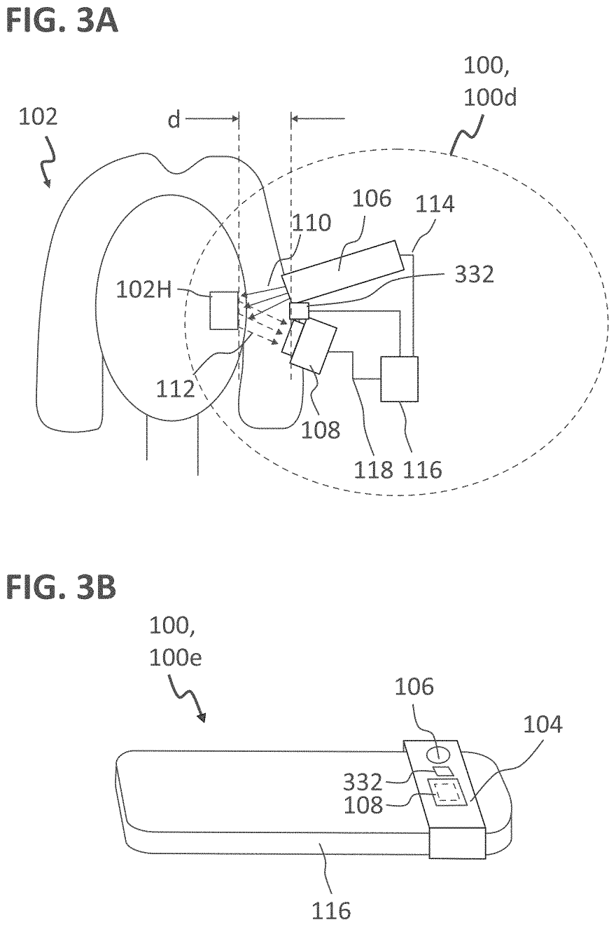

FIG. 1A, FIG. 1B, FIG. 2, FIG. 3A and FIG. 3B respectively show a device 100 (100a, 100b, 100c, 100d, or 100e) for determining a degree of bacterial colonization of a user's skin in accordance with various exemplary embodiments.

A user's skin of a user 102 (FIG. 1A, FIG. 1B and FIG. 3A show a region of skin 102H by way of example) may be colonized by bacteria. The bacteria may also include acne bacteria, for example Propionibacterium acne. Even though a plurality of different bacteria can typically colonize the skin of a human being, the method and devices in accordance with various exemplary embodiments described herein will concern only those bacteria which are capable of causing skin blemishes, redness and inflammation (for example all the way to acne) and which include (for example form) a porphyrin (for example coproporphyrin III and/or protoporphyrin IX). This means that terms such as the “degree of bacterial colonization of skin” and the like as used herein refer only to the (acne) bacteria described, and not to colonization with any species of bacteria.

In various exemplary embodiments, the device 100 for determining a degree of bacterial colonization of the user's skin may have a camera 108, which may be configured to record an image of the region of skin 102H, whereupon an image of the region of skin 102H can be produced. The camera 108 may, for example, be a digital camera which records an image of the region of skin 102H captured by it. In various exemplary embodiments, instead of the camera 108, another light measuring device may be used which provides, for the region of skin 102H captured by it, only an integrated signal relating to the area, for example a photometer. For the purposes of simplification below, the term “camera” will be used even if in many exemplary embodiments (apart from those in which special forms of representations of the results are used, in which the imaging property of the camera is used, see below), the other light measuring device may also be used instead of the (imaging) camera.

In various exemplary embodiments, the camera 108 may be configured to at least capture light in a fluorescence wavelength range of a fluorescent agent produced by the acne bacteria. As an example, the camera 108 may have a detector which can receive at least in the fluorescence wavelength range of the fluorescent agent produced by the acne bacteria. The fluorescent agent may be or comprise a porphyrin, for example coproporphyrin III and/or protoporphyrin IX. The fluorescence wavelength range, i.e. the wavelength range in which the fluorescent agent emits fluorescent light, may be in a range from approximately 590 nm to approximately 760 nm, for example between approximately 600 nm and approximately 740 nm, i.e. in a yellow-red to red or pure red wavelength range of visible light.

In various exemplary embodiments, the device 100 for determining a degree of bacterial colonization of the user's skin may comprise an illuminating device 106.

In various exemplary embodiments, the illuminating device 106 may be configured to illuminate the at least one region of skin 102H of the user 102 with ultraviolet and/or blue light 110. A wavelength or a wavelength range of ultraviolet and/or blue light 110 may be selected in a manner such that there is at least an overlap with an excitation wavelength for the fluorescent agent (for example the porphyrin) for fluorescence. The wavelength of the ultraviolet and/or blue light 110 emitted from the illuminating device may, for example, be in a wavelength range between approximately 315 nm and approximately 450 nm, for example between approximately 315 nm and approximately 380 nm, for example between approximately 340 nm and approximately 380 nm or, for example, between approximately 355 nm and approximately 380 nm.

In various exemplary embodiments, as shown diagrammatically in FIG. 1A, FIG. 1B and FIG. 3A, the illuminating device 106 and the camera 108 may be disposed relatively to each other and to the region of skin 102H in a manner such that the illuminating device 106 can illuminate the region of skin 102H with the ultraviolet and/or blue light 110 which there (in/with the acne bacteria which might be present) can excite any porphyrin present to emit fluorescent light 112, and at least a portion of the fluorescent light 112 is captured by the camera 108, i.e. at least a portion of the fluorescent light 112 reaches the detector of the camera 108 and is captured there.

In various exemplary embodiments, the device 100 for determining a degree of bacterial colonization of the user's skin may further comprise electronic circuitry 116. In various exemplary embodiments, the electronic circuitry 116 may be configured to determine, by the image, a captured quantity of light which is emitted from the fluorescent agent as fluorescent light 112 as a consequence of the illumination (this quantity of light may also be described herein as the fluorescence signal), and to allocate a degree of colonization of the skin to the determined captured quantity of light by a database, wherein the database can provide an allocation of reference quantities of light and known degrees of bacterial colonization of the skin, for example for a comparison with the fluorescence signal.

As can be seen in FIG. 1A, FIG. 1B and FIG. 3A as the region of skin 102H, in various exemplary embodiments, the region of skin 102H may be a part of the skin of the user's face, which typically may be the most affected by blemishes, redness, inflammation, etc. Alternatively or in addition, another or further region of skin, for example on the user's back, may be used to determine the degree of colonization with bacteria.

In various exemplary embodiments, the illuminating device 106 and the camera 108 may be coupled with the electronic circuitry 116, for example by a (cabled or cordless) data link 114 or 118.

The determination, by the image, of a captured quantity of light which is emitted from the fluorescent agent as fluorescent light 112 as a consequence of the illumination may in various exemplary embodiments comprise using a measured value provided by the camera 108 as the light quantity value (fluorescence signal) representing the quantity of light, for example in a case in which the camera 108 is an integrated light measuring device which is configured, for example by a filter 104 disposed between the region of skin 102H and the camera 108 (see FIG. 2 and FIG. 3B), which is only transparent to light from a wavelength range in which the fluorescence typically significantly predominates over other light inputs from the region of skin 102H, for example in a proportion of at least about 75% (for example in a range from approximately 640 nm to approximately 650 nm), and/or in a case in which other light inputs from the region of skin 102H are minimized or eliminated, for example by carrying out the method in darkness or near-darkness (i.e. when no or almost no light, apart from the ultraviolet or blue light 110 from the illuminating device 106, illuminates the region of skin 102H).

The determination, by the image, of a captured quantity of light which is emitted from the fluorescent agent as fluorescent light 112 as a consequence of the illumination may in various exemplary embodiments comprise integration of measured intensity values over a surface region in an image provided by the camera 108 (for example over the entire image), for example summing of picture element values, and using the integrated value as the light quantity value (fluorescence signal) representing the quantity of light, for example in a case in which the camera 108 is an imaging (digital) camera which is configured in a manner such that, for example by a filter 104 disposed between the region of skin 102H and the camera 108 (see FIG. 2 and FIG. 3B), which is only transparent to light from a wavelength range in which the fluorescence typically significantly predominates over other light inputs from the region of skin 102H, for example in a proportion of at least about 75% (for example in a range from approximately 640 nm to approximately 650 nm), and/or in a case in which other light inputs from the region of skin 102H are minimized or eliminated, for example by carrying out the method in darkness or near-darkness (i.e. when no or almost no light, apart from the ultraviolet or blue light 110 from the illuminating device 106, illuminates the region of skin 102H).

The acne bacteria typically colonize pores of the skin so that the fluorescence emitted from the porphyrin also appears to originate from the colonized pores of the skin, so that in a recorded image, with colonized skin, the colonized pores of the skin may be shown up as punctiform or essentially circular regions with enhanced emission. Correspondingly, in various exemplary embodiments, the surface area over which the measured intensity values are integrated (for example summed), may be composed of the punctiform or essentially circular regions with the enhanced emission.

The determination, by the image, of the captured quantity of light which is emitted from the fluorescent agent as fluorescent light 112 as a consequence of the illumination may in various exemplary embodiments furthermore comprise a determination of the other light inputs from the region of skin 102H and subtracting the other light inputs from the integrated intensity values.

The determination of the other light inputs from the region of skin 102H may in various exemplary embodiments comprise recording a background light image, as can be seen in FIG. 1B (top). When recording the background light image, the illuminating device 106 may be switched off. To subtract the other light inputs from the integrated intensity values, the background image may be integrated over the same surface area as the image, for example over the entire image or, for example, over the punctiform or essentially circular regions in which the pores of the skin with the acne bacteria are depicted.

The determination of the other light inputs from the region of skin 102H may in various exemplary embodiments (not shown) comprise determining the other light inputs from the image, for example by integrating only the punctiform or essentially circular regions in which the pores of the skin with the acne bacteria are depicted (for example, a circle outside the punctiform or essentially circular regions in which no more fluorescence emission is present), and an intensity value obtained from these regions (which in total may have a different area, for example another number of picture elements than in the totaled punctiform or essentially circular regions) by integration (for example summing) may be normalized in a manner such that it corresponds to the surface area of the punctiform or essentially circular regions, and the normalized intensity value of the integrated value can be subtracted from the punctiform or essentially circular regions. In various exemplary embodiments, this method may also be carried out individually for each punctiform or essentially circular region and the difference between the punctiform or essentially circular regions with respect to the determined quantity of light may be summed.

In various exemplary embodiments, for example as an alternative to using a special filter 104 or in addition thereto, when using a digital colour camera, only a red fraction of the intensity value may be used for the determination of the captured quantity of light which has been emitted from the fluorescent agent as fluorescent light 112 as a consequence of the illumination. As an example, when (as is usual with digital colour cameras), encoding is carried out in a RGB colour space, an assessment of the intensity value may be limited to a R channel (the red colour channel). Similarly, when using other colour spaces, the colour channel for the determination of the captured quantity of light which has been emitted from the fluorescent agent as fluorescent light 112 as a consequence of the illumination may be used, in which the fluorescence wavelength range (i.e. the region between approximately 560 nm and approximately 780 nm) predominantly lies.

In various exemplary embodiments, the electronic circuitry 116 may be configured, with the aid of the determined quantity of light, for example by comparison with a database which may comprise the allocation of reference quantities of light to known degrees of bacterial colonization of the skin, to determine a degree of bacterial colonization of the skin of the user 102, for example a very high, moderately high, normal, moderately low or very low degree of bacterial colonization of the skin. In various exemplary embodiments, the degree of bacterial colonization of the skin may be described or quantified in other manners, for example as a numerical degree of bacterial colonization (for example with any units) or the like.

In various exemplary embodiments, the electronic circuitry 116 may be further configured to determine a dimension (i.e. a physical dimension, for example a length or a width, a diameter or the like in a unit of length, for example centimetres) of the captured region of skin 102H.

In various exemplary embodiments, the electronic circuitry 116 may be configured to determine the dimension of the captured region of skin 102H with the aid of the image, for example with the aid of a dimension of the essentially circular regions, as those which could depict the bacterially colonized pores of the skin (as long as they are depicted in a spatially resolved manner), comparing them with typical dimensions of pores of the skin, with the aid of a mean distance between a plurality (for example adjacent) of the punctiform or essentially circular regions, as those which could depict the bacterially colonized pores of the skin, comparing them with typical pore distances, and/or with the aid of other structures of the face which, for example, can be recognised using conventional methods in the image, comparing them with typical values for the structures, for example a diameter of the iris (which is relatively constant in human beings, at approximately 12 mm), a distance between pupils and/or the nostrils of the user 102, the tip of the nose-to-chin distance, or similar structures.

In various exemplary embodiments, the device 100 for determining a degree of bacterial colonization of the user's skin as shown, for example, in FIG. 3A and FIG. 3B for the devices 100d or 100e, may comprise a distance measuring device 332 for measuring a distance d between the distance measuring device 332 and the region of skin 102H. In this case, the electronic circuitry 116 may be configured to determine, by the image, the captured quantity of light which has been emitted from the fluorescent agent as fluorescence as a consequence of the illumination, determining the (physical) size of the region of skin 102H taking camera properties into account, the measured distance d between the distance measuring device 332 and the region of skin 102H and a distance (parallel to the camera-region of skin direction) between the distance measuring device 332 and the camera 108, and allocating a degree of colonization of the skin to the determined captured quantity of light by a database, taking the dimension of the region of skin 102H into account.

The camera 108 properties may be a magnification and physical measurements of the detector (for example length and width in millimetres), or other appropriate combinations of camera properties which enable the dimension of the region of skin 102H to be calculated by including the distance between the camera 108 and the region of skin 102H.

In various exemplary embodiments, as the distance measuring device 332, a conventional triangulation device, for example using a light emitting diode and a detector, may be used, or another suitable distance measuring device. In various exemplary embodiments, for example when using a smartphone, tablet or iPad etc with an integrated camera, the integrated camera as well as the camera 108 may be used, for example, and also, if appropriate, information provided by the autofocussing system of the camera 108 for determining the distance between the camera 108 and the region of skin 102H.

In various exemplary embodiments, the distance between the camera 108 and the region of skin 102H may only be determined if an image (to be evaluated) is present. In various exemplary embodiments, the distance between the camera 108 and the region of skin 102H may be continuously determined, while the device 100 for determining a degree of bacterial colonization of the user's skin is being used. In this manner, the distance determination may be used, for example, to instruct the user 102 to find a preferred distance, for example by an optical signal, as an example, the illuminating device 106 could be operated at intervals, at a frequency which changes as the preferred distance is approached, and is operated constantly when the preferred distance has been (for example approximately) reached, or, for example by an acoustic signal which, for example, sounds less frequently as the preferred distance is approached and falls silent when the preferred distance has been (for example approximately) reached.

Alternatively or in addition to the distance measuring device and/or to determining the distance with the aid of the image, the device 100 may be provided with a spacer (not shown). By the spacer, a preferred distance between the camera 108 and the region of skin 102H may be set, so that when the camera properties are included, the (physical) dimension of the region of skin 102H can be determined without measuring the distance between the camera 108 and the region of skin 102H or the dimensions of depicted structures.

In various exemplary embodiments, a knowledge of the (physical) dimensions of the region of skin 102H may allow for an improved quantification of the degree of bacterial colonization to be carried out, for example because the determined captured quantity of light is set in relationship to the dimensions of the region of skin 102H, for example as a quantity of light per square centimetre, and/or because, by comparing the quantity of light with the plurality of reference quantities of light, the physical size of the region of skin 102H is taken into account.

In various exemplary embodiments, the database may comprise a plurality of reference quantities of light and a respectively allocated degree of bacterial colonization, which may be determined at least in part by laboratory measurements and can all be normalized taking the physical dimensions of the reference region of skin 102H into account with respect to a unit area (for example as the quantity of light per square centimetre or the like), wherein values for comparable degrees of colonization may be determined independently of how large the reference region of skin 102H is.

In various exemplary embodiments, qualitative descriptions may be used as the degrees of colonization, for example “severely colonized”, “weakly colonized” etc. In various exemplary embodiments, alternatively or in addition, quantitative degrees of colonization may be used which, for example, may be determined by laboratory measurements, for example an average bacterial count per square centimetre or the like.

In various exemplary embodiments, in addition to a plurality of reference quantities of light and the respectively allocated degree of bacterial colonization which, for example, may be at least in part determined by laboratory measurements, a respective (physical) dimension (for example in square centimetres or square millimetres) for the reference region of skin 102H may be given in the database for which the reference quantity of light has been determined, and for the comparison of the quantity of light with the reference quantity of light for determining the associated degree of bacterial colonization, the physical dimensions of the region of skin 102H can be taken into account, for example only those inputs in the database (i.e. allocations of quantity of light to degree of bacterial colonization) can be called upon for comparison which have allocations based on regions of skin 102H with similar dimensions. This makes it possible to take into consideration the fact that a region of usually measurable quantities of light for a given degree of bacterial colonization may be dependent on the (physical) dimensions of the region of skin 102H. As an example, even with a very high degree of colonization, it may not be possible when determining the quantity of light per unit area for, for example, an entire face, to determine values which are as high as when the quantity of light per unit area is determined for only, for example, a one square centimetre-sized region of skin 102H on the chin (because some regions of skin are typically, even with a severe degree of bacterial colonization, free from bacteria or low in bacteria, for example in regions under the eyes), and vice versa.

In various exemplary embodiments, the device 100 for determining a degree of bacterial colonization of a user's skin may be a portable device 100 as shown by way of example in FIG. 2 and FIG. 3B, wherein the portable device 100 is not limited to a smartphone.

In the electronic circuitry 116 may comprise a processor and a memory. In various exemplary embodiments, the electronic circuitry 116 may be coupled to the camera 108 and the illuminating device 106, for example in a manner such that transmission of a signal captured with the camera (for example an image, for example a digital image or a value in a photometer) is possible, for example by a data connection 118. The data connection 118 may, for example, be a data cable, an internal conducting connection and/or a cordless transmission, for example by WLAN. Thread, ZigBee or Bluetooth. The electronic circuitry 116 may, for example, be a smartphone, an iPad, a tablet, a laptop or the like.

In various exemplary embodiments, (as can be seen, for example, in FIG. 2 and FIG. 3B for the devices 100c or 100e), the camera 108 and the electronic circuitry 116 may form a portable integrated unit.

In various exemplary embodiments, the illuminating device 106 and the camera 108 may form a portable integrated unit.

In various exemplary embodiments, the device 100 has dimensions which enable it to be accommodated in a handbag or in a trouser pocket. As an example, the device 100 may have a surface area of less than 36 cm² and a thickness of less than 2 cm.

In various exemplary embodiments, the electronic circuitry 116 may be configured to determine a degree of bacterial colonization of the skin as described above. In various exemplary embodiments, the electronic circuitry 116 may be configured to determine the degree of bacterial colonization of the skin by itself, i.e. directly.

In various exemplary embodiments, to determine the degree of bacterial colonization of the skin, software, for example an app, may be used which, for example, may be installed on a portable data processing device, wherein the portable data processing device may, for example, be the electronic circuitry 116 as can be seen, for example, in FIG. 2 and in FIG. 3B, or it may be an additional, for example external, data processing device (not shown; as an example, the camera 108 and the illuminating device 106 may form an integrated unit which may be coupled to the external data processing device, for example a smartphone, a tablet, an iPad or the like).

In various exemplary embodiments, the degree of bacterial colonization of the skin may be determined as a function of time (for example several times daily, daily, weekly, monthly or with any other temporal dependency) and/or under different environmental conditions (for example in an environment with higher or lower humidity, when cold or in the heat, under stronger or weaker sunlight etc).

In various exemplary embodiments, the reference measurements which, for example, may be provided in the form of a database, may be used as part of the software/app, in order to allocate the determined captured quantity of light to a degree of bacterial colonization of the skin.

In various exemplary embodiments, the database may be stored in the memory of the electronic circuitry 116.

In various exemplary embodiments, the database may be stored in an external data processing device, for example in cloud computing architecture 222. In this case, the database may be provided to the electronic circuitry 116 for the comparison with the determined captured quantity of light, and/or, after providing the captured quantity of light or at least the image and optionally further data (for example the data described above for determining the distance between the camera and the region of skin 102H), the comparison may be carried out by the cloud computing architecture. This may be described as indirect determination of the degree of bacterial colonization by the electronic circuitry 116. The degree of bacterial colonization determined by the comparison carried out by the external data processing device 222 may be provided to the electronic circuitry 116, for example for provision to the user 102.

In order to transmit/provide data between the external data processing device 222, for example the cloud computing architecture 222 and the electronic circuitry, a data connection 224 may be used, for example a cordless data connection 224, for example by WLAN or Bluetooth or a comparable cordless data transmission device. In order to provide and receive data to/from the external data processing device 222, the device 100 may comprise a data exchange device 120 (see FIG. 1B) which, for example, may be part of the electronic circuitry 116 or may be connected thereto.

In various exemplary embodiments, for example in a case in which the database is stored in cloud computing architecture 222, the database may be updated using data from other users 1020 as can be seen, for example, in FIG. 1A and FIG. 1B.

In various exemplary embodiments, the electronic circuitry 116 may comprise a relatively simple electronic circuitry which, for example, may be configured to essentially capture only the camera signal and to transmit it to the external data processing device 222. In various exemplary embodiments, the electronic circuitry 116 may be configured as higher performance, multifaceted circuitry (for example a smartphone, a tablet or an iPad) which may not only be configured to capture the camera signal (and optionally to determine the captured quantity of light and compare it with the reference quantities of light in the database and/or to transmit it to the external data processing device 222), but may additionally be configured to carry out various other functions, for example to request and store inputs from the user 102 and to show results, values (for example autofocus values) for determining the distance from the camera to the region of skin, etc.

In various exemplary embodiments, the degree of bacterial colonization of the user's skin may be provided by the electronic circuitry 116, for example by the software/app. The degree of bacterial colonization of the skin may, for example, be communicated as a value (for example as described above), as a voice message, as a graphical representation or the like, for example displayed, for example by a display, for example on a screen of the portable electronic circuitry 116.

In various exemplary embodiments, the provision of the skin condition to the user by the graphical representation may comprise the image on which an illustration of the degree of bacterial colonization of the skin may be overlaid, for example by different symbols/marks/shading for the regions with different degrees of bacterial colonization of the skin.

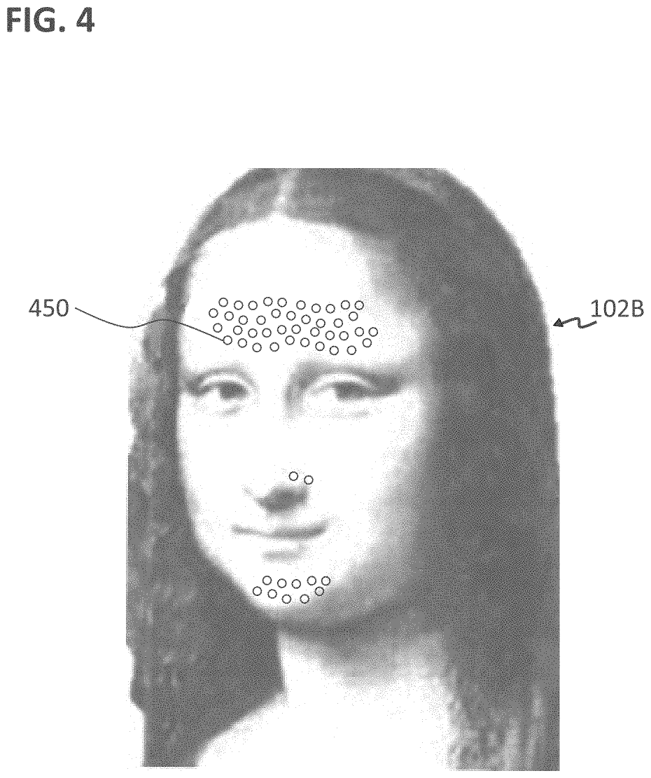

As can be seen in FIG. 4, to illustrate the degree of bacterial colonization of the skin, the image 102B may be used which, for example by the electronic circuitry 116 and/or the external data processing device 222, may be used in order, for example, to produce a two-dimensional intensity distribution of the determined captured fluorescent light in the region of skin 102H, for example a two dimensional digital image which comprises the measurements of the image, but only at locations for which a fluorescent light contribution has been determined for the determined intensity values, which comprises the fluorescent light contribution as an intensity value. The intensity distribution of the determined fluorescent light may, for example, be overlaid on the image 102B as a contour map 450 (as can be seen by way of example in FIG. 4) and/or as a false colour representation, for example on a screen of the electronic circuitry 116.

In various exemplary embodiments, intensity distributions which have been determined at different points in time may be represented in different manners, for example with different colours.

In various exemplary embodiments, the device 100 may be further configured, based on the determined degree of bacterial colonization of the skin, to determine a cosmetic skin treatment recommendation and to provide it to the user 102.

In various exemplary embodiments, each of the plurality of degrees of bacterial colonization of the skin may be allocated at least one suitable cosmetic product and/or at least one care tip. The allocation may, for example, have been determined experimentally, for example in laboratory tests. In this respect, the cosmetic product and/or the care tip for the skin may be suitable for the skin with the given degree of bacterial colonization when it is either suitable for reducing the degree of bacterial colonization or, when another objective is in sight, for example for a predominantly decorative use, at least not to increase the degree of bacterial colonization.

In various exemplary embodiments, data from the literature may be consulted in order to assess a suitability of a care product and/or a care tip for care of a skin with a given degree of bacterial colonization of the skin.

In various exemplary embodiments, as can be seen in FIG. 1B, an assessment of a suitability of a care product for reducing a degree of bacterial colonization of the skin may be confirmed or modified by recording empirical values from other users 1020 with the same or a similar degree of bacterial colonization of the skin, for example empirical values regarding the success of a treatment. The empirical values may, for example, be provided from the other users, for example to the external data processing device 222, for example by a cordless data transmission 226. Alternatively, transmission of data by cable may be employed. The database in the external data processing device 222 and/or in the portable electronic circuitry 116 may be updated with the aid of the empirical values. In this manner, it may be possible for the user 102 to obtain constantly more optimized recommendations.

In various exemplary embodiments, when determining the product and/or care recommendations, further information regarding the general state of health, a skin condition, eating habits and other behavioural aspects of the user (for example time spent daily outdoors/in the sun/in water (for example in water containing trace elements), smoking habits etc) may be used, for example by the software/app installed on the portable electronic circuitry 116 and/or on the external data processing device 222. In various exemplary embodiments, the information may be requested from the user 102 using the portable electronic circuitry 116 which they can input into the electronic circuitry 116, for example by a keyboard, as a voice command, by selection from a menu displayed by the portable electronic circuitry 116, or the like.

As an example, when the user 102 provides the additional information that they spend a lot of time in water or outdoors, when recommending the product and/or care tips based on the degree of bacterial colonization of the skin of the user 102, allocated care products which, for example, are recommended to the user 102 may be those which are not only suitable for their degree of bacterial colonization, but are also, for example, waterproof and/or provided with a UV screen.

In various exemplary embodiments, the product recommendation and/or the care tip may be provided to the user 102, for example by the electronic circuitry 116, for example by the screen of the electronic circuitry 116. As an example, the graphical representation of FIG. 4 may be supplemented by messages (for example text messages) (not shown), for example the determined product recommendations and/or care tips such as, for example, “Wear cream A in the zone marked by the contour 450” or the like.

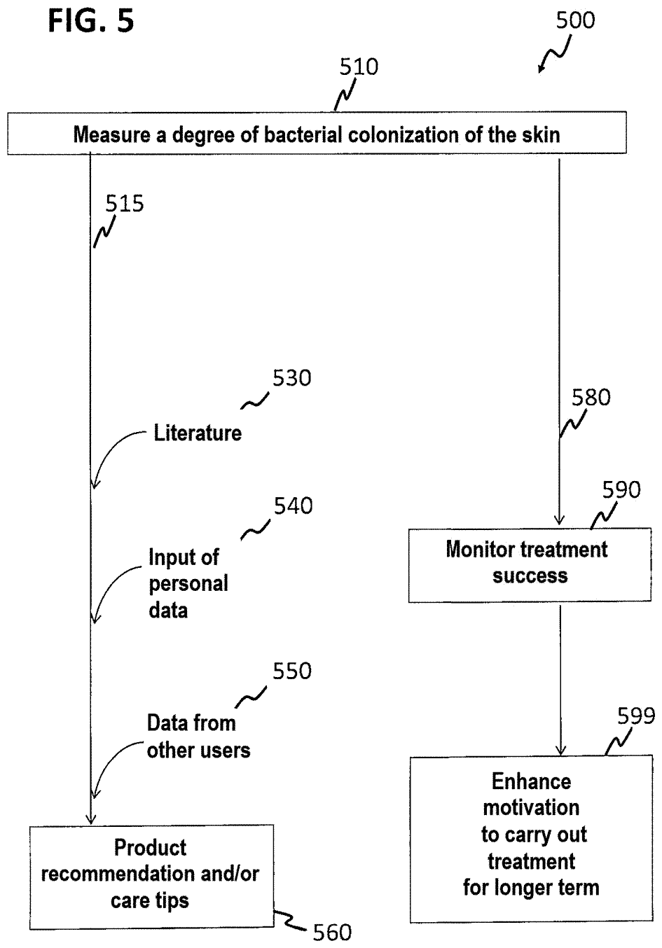

FIG. 5 shows a diagram 500 to illustrate a method for determining a cosmetic product for skin treatment in accordance with various exemplary embodiments. FIG. 5 can essentially illustrate those processes which have been described elsewhere in connection with the method for determining a cosmetic product for skin treatment in accordance with various exemplary embodiments.

In accordance with various exemplary embodiments, the method may comprise determining a degree of bacterial colonization of the skin (at 510), for example as described above.

Based on the determined degree of bacterial colonization of the skin, a product or care tip recommendation may be determined (at 560). In various exemplary embodiments, (shown as path 515), in addition to the degree of bacterial colonization of the skin, data from the literature (indicated at 530), personal data (for example as described above, indicated at 540) and/or data from other users, for example their empirical values (indicated at 550) may be consulted.

In various exemplary embodiments, furthermore, as can be seen in pathway 580, by determining the degree of bacterial colonization of the skin (at 510), the success of a treatment may be monitored (at 590). This may in particular, for example, be useful during and after a cosmetic treatment based on the product recommendation (at 560). A successful treatment using the method for determining the skin condition with the aid of objective values may enhance the motivation of the user (at 599).

In various exemplary embodiments, as described above, with the aid of the determined degree of bacterial colonization of the skin, product recommendations for suitable cosmetic products which are tailored to the user 102 and/or individual care tips may be derived. The product recommendations and/or care tips may, for example, be provided by software, for example an app.

The user may be prompted to buy a recommended product directly and the user can trigger the purchase via an input. In addition to purchasing recommended products, further information regarding the offers may be presented to the user. This further information may concern detailed treatment and indications for use. Software or an app might, for example, receive the request that the user might want to purchase the product, it stores the request and/or transmits the request to a business that markets the product. By the software/app, the user may be requested to provide their personal data (address, bank information, shipping preferences, etc) via the input unit. Alternatively, the user could be informed as to where (for example a drug store, beauty salon, pharmacy, etc) they can obtain the recommended product. As an example, the software/app could be the very same software or app which carries out the method for determining a cosmetic skin treatment recommendation.

In various exemplary embodiments, the user may be encouraged to use cosmetic products which are individually produced for the individual and an ordering procedure, preferably by calling up an internet site of the manufacturer of individual cosmetic products, may be introduced.

More and more customers want a product that is individually tailored to their needs. In this regard, this may be a product that is manufactured specifically for the customer, or what is known as a “mass customized” product. A “mass customized” product can be individualized by modifying just a few features of a product which are nevertheless seen by the customer as important. Preferably, these “mass customized” products are based on the concept of modularization, which means that the product can be individually put together from a variety of modules/components.

Among the many different features/ingredients of a product, there are frequently many dependencies which may be expressed as “goodies” or “baddies”. In order to obtain a unique product definition, it may be advantageous for the ordering procedure to run with the aid of a product configurator. This configurator aids the customer in their choice of features/ingredients and advises them of permissible/non-permissible combinations of features, wherein the latter cannot be selected.

With products for the skin, the relevant product features in particular include the chemical ingredients of the substance, the physical properties of the substance and the type of manufacture of the agent. With the aid of a product configurator, for example, the selection of chemically and/or physically incompatible ingredients or the selection of ingredients which are unsuitable for the determined degree of colonization can be avoided. In contrast, the selection of ingredients which are suitable for the determined degree of colonization can be highlighted or suggested by the product configurator.

In various exemplary embodiments, the software/app which determines the degree of bacterial colonization of the skin may be the same as that which determines the product recommendation and/or the care tips. In various exemplary embodiments, different software programs/apps may be used for the various procedures (determining the degree of bacterial colonization of the skin, determining a product recommendation, determining a care tip).

In various exemplary embodiments, it may be recommended that a dermatologist or beautician be consulted. In various exemplary embodiments, a booking procedure may be initiated directly via the software/app which determines the degree of colonization. In this regard, for example, the software/app may store the contact details of dermatologists and/or beauticians and display them to the user. In addition, the selection can be limited by filters such as, for example, a post code. In various exemplary embodiments, an appointment may be booked directly via the software/app. Alternatively, a dermatologist and/or beautician appointment may be made via separate software/apps, for example Treatwell.

In various exemplary embodiments, the success of a cosmetic treatment which may be intended to have a positive influence on the determined degree of bacterial colonization of the skin may be monitored. In various exemplary embodiments, the software/app may allow monitoring and/or follow-up of the results by a representation (for example a graphical representation) of the measurement results over time.

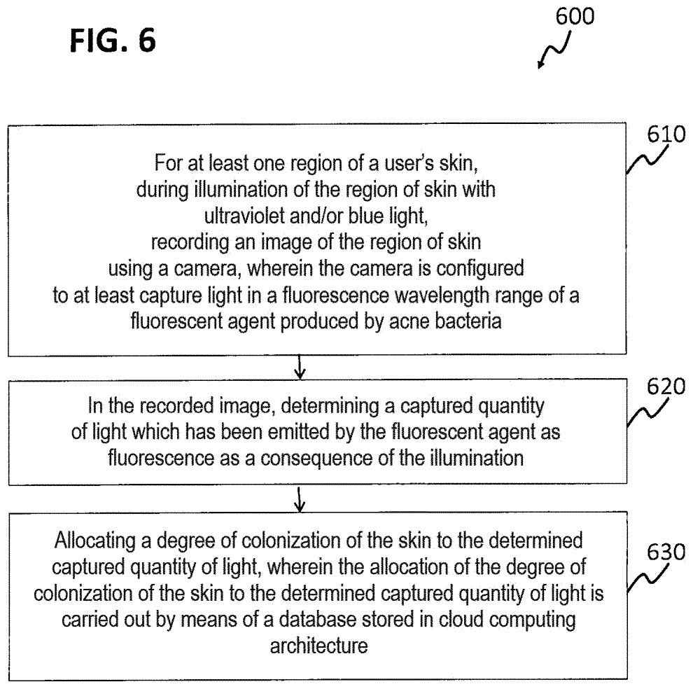

FIG. 6 shows a flow diagram 600 for a method for determining a degree of bacterial colonization of a user's skin in accordance with various exemplary embodiments.

In various exemplary embodiments, a method for determining a degree of bacterial colonization of a user's skin is provided. For at least one region of a user's skin, during illumination of the region of skin with ultraviolet and/or blue light, the method can record an image of the region of skin using a camera, wherein the camera is configured to at least capture light in a fluorescence wavelength range of a fluorescent agent produced by acne bacteria (at 610), in the recorded image, determine a captured quantity of light which has been emitted by the fluorescent agent as fluorescence as a consequence of the illumination (at 620), and allocate a degree of colonization of the skin to the determined captured quantity of light, wherein the allocation of the degree of colonization of the skin to the determined captured quantity of light is carried out by a database stored in cloud computing architecture (at 630).

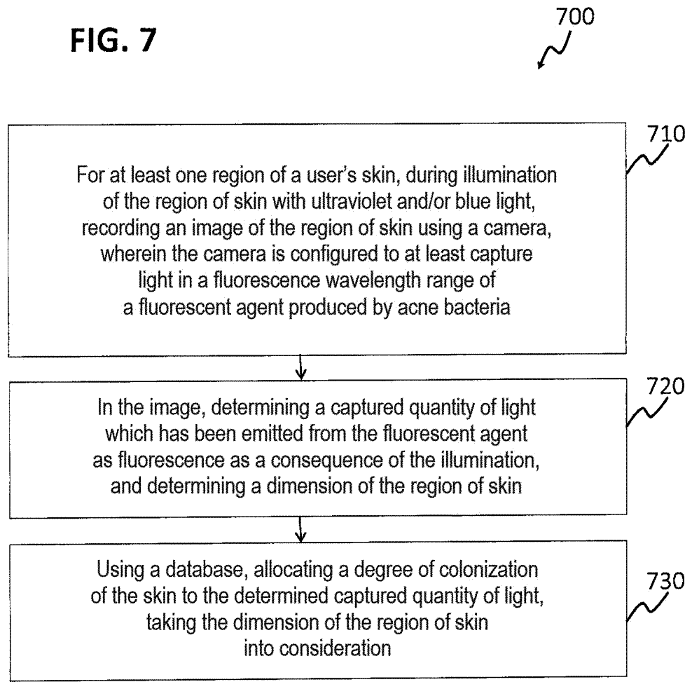

FIG. 7 shows a flow diagram 700 for a method for determining a degree of bacterial colonization of a user's skin in accordance with various exemplary embodiments.

In various exemplary embodiments, a method for determining a degree of bacterial colonization of a user's skin is provided. For at least one region of a user's skin, during illumination of the region of skin with ultraviolet and/or blue light, the method can record an image of the region of skin using a camera, wherein the camera is configured to at least capture light in a fluorescence wavelength range of a fluorescent agent produced by acne bacteria (at 710), in the recorded image, determine a captured quantity of light which has been emitted from the fluorescent agent as fluorescence as a consequence of the illumination, and determine a dimension of the region of skin (at 720), and, using a database, allocate a degree of colonization of the skin to the determined captured quantity of light, taking the dimension of the region of skin into consideration (at 730).

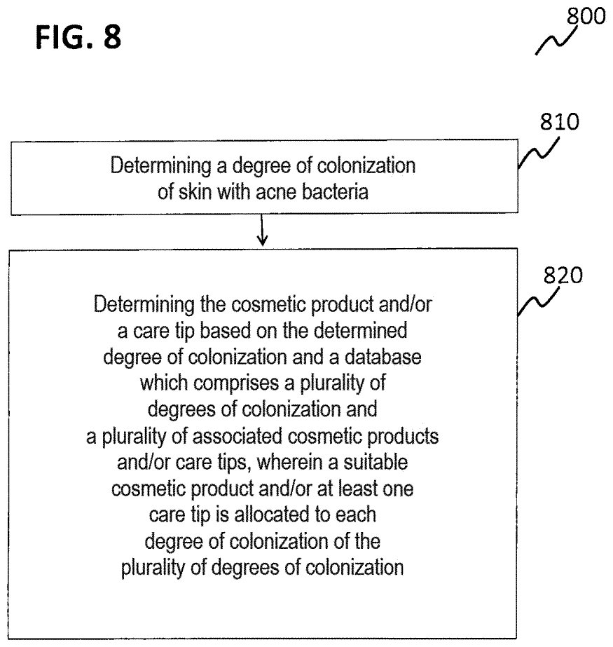

FIG. 8 shows a flow diagram 800 for a method for determining a cosmetic skin treatment recommendation in accordance with various exemplary embodiments.

In various exemplary embodiments, a method for determining a cosmetic skin treatment recommendation is provided. The method may include: determining a degree of bacterial colonization of a user's skin in accordance with various exemplary embodiments (at 810) and determining the cosmetic product and/or the care tips based on the determined skin condition and a database which comprises a plurality of skin conditions and a plurality of associated cosmetic products or care tips, wherein at least one suitable cosmetic product and/or at least one suitable care tip may be allocated to each degree of bacterial colonization of the plurality of degrees of bacterial colonization (at 820)

Some of the exemplary embodiments have been described in connection with devices, and some of the exemplary embodiments have been described in connection with methods. Further advantageous implementations of the method can be obtained from the description of the device, and vice versa.

A first preferred embodiment of the present disclosure comprises a method for determining a degree of colonization of skin with acne bacteria, comprising:
for at least one region of a user's skin, during illumination of the region of skin with ultraviolet and/or blue light, recording an image of the region of skin using a camera, wherein the camera is configured to at least capture light in a fluorescence wavelength range from a fluorescent agent produced by the acne bacteria;
in the image, determining a captured quantity of light which is emitted from the fluorescent agent as fluorescence as a consequence of the illumination, and;
allocating a degree of colonization of the skin to the determined captured quantity of light, wherein the allocation of the degree of colonization of the skin to the determined captured quantity of light is carried out by a database stored in cloud computing architecture.

A second preferred embodiment includes the feature that the method in accordance with embodiment 1 furthermore comprises updating the database based on new allocations of the degree of colonization of the skin to the determined captured quantity of light from a plurality of other users.

A third preferred embodiment includes the feature that the method in accordance with embodiment 1 or 2 furthermore comprises recording a background light image of the region of skin using the camera while the region of skin is not illuminated with the ultraviolet and/or blue light, wherein the determination of the captured quantity of light which is emitted from the fluorescent agent as fluorescence as a consequence of the illumination, comprises a determination of a difference between the image and the background light image.

A fourth preferred embodiment comprises the method in accordance with one of embodiments 1 to 3, wherein the fluorescent agent comprises a porphyrin, preferably coproporphyrin III.

A fifth preferred embodiment comprises the method in accordance with one of embodiments 1 to 4, wherein the fluorescence wavelength range is from 560 nm to 780 nm.

A sixth preferred embodiment comprises the method in accordance with one of embodiments 1 to 5, wherein the at least one region of skin comprises a plurality of regions of the user's skin.

A seventh preferred embodiment comprises the method in accordance with one of embodiments 1 to 6, wherein the camera and an illuminating device for producing the ultraviolet and/or blue light are parts of at least one portable device.

An eighth preferred embodiment comprises the method in accordance with one of embodiments 1 to 7, wherein the portable device comprises a smartphone, a tablet or an iPad or is capable of being attached to a smartphone, a tablet or an iPad.

A ninth preferred embodiment comprises the method in accordance with one of embodiments 1 to 8, wherein the determination of the captured quantity of light and/or the allocation of the degree of colonization of the skin to the determined captured quantity of light is carried out by an app.

A tenth preferred embodiment comprises a method for determining a cosmetic skin treatment recommendation, comprising:
determining a skin condition in accordance with one of claims 1 to 9; and
determining the cosmetic product and/or a care tip based on the determined degree of colonization of the skin and a further database stored in the cloud computing architecture which comprises a plurality of degrees of colonization and a plurality of associated cosmetic products and/or care tips, wherein each skin condition of the plurality of skin conditions is associated with at least one suitable cosmetic product and/or at least one care tip.

An eleventh preferred embodiment comprises the method in accordance with embodiment 10, wherein the cosmetic product and/or the care tips is suitable for the degree of colonization of the skin when, based on empirical values stored in the further database from a plurality of other users of the cosmetic product, a reduction in the degree of colonization of the skin is to be expected.

A twelfth preferred embodiment comprises the method in accordance with embodiment 10 or 11, wherein the determination of the degree of colonization of the skin and/or the determination of the cosmetic skin treatment recommendation comprises transmitting the image and/or the degree of colonization of the skin to an external data processing device and receiving the degree of colonization of the skin and/or the cosmetic skin treatment recommendation.

A thirteenth preferred embodiment comprises a device for determining a degree of colonization of skin with acne bacteria, comprising:
an illuminating device for illuminating at least one region of a user's skin with ultraviolet and/or blue light;
a camera to record an image of the region of skin during the illumination of the region of skin by the illuminating device, wherein the camera is configured to at least capture light in a fluorescence wavelength range of a fluorescent agent produced by the acne bacteria;
electronic circuitry which is configured to determine, in the image, a captured quantity of light which is emitted from the fluorescent agent as fluorescence as a consequence of the illumination, and to allocate a degree of colonization of the skin to the determined captured quantity of light by a database; and
a data exchange device for exchanging data between the electronic circuitry and cloud computing architecture, wherein by the data exchange device, the database stored in the cloud computing architecture is made available for the allocation of the degree of colonization to the quantity of light.

A fourteenth preferred embodiment comprises a device in accordance with embodiment 13, wherein the fluorescence wavelength range is from 560 nm to 780 nm.

A fifteenth preferred embodiment comprises a device in accordance with embodiment 13 or 14, wherein the ultraviolet and/or blue light is in a wavelength range of between about 380 nm and about 500 nm.

A sixteenth preferred embodiment comprises a device in accordance with embodiment 13 or 14, wherein the ultraviolet and/or blue light is in a wavelength range of between about 315 nm and about 380 nm.

A seventeenth preferred embodiment comprises a device in accordance with one of embodiments 13 to 16, further comprising: a filter the transmittance of which is configured and which is disposed in a manner such that the ultraviolet and/or blue light of the illuminating device is prevented from being captured by the camera, whereas the filter is transparent to the light in the fluorescence wavelength range.

An eighteenth preferred embodiment comprises a device in accordance with one of embodiments 13 to 17, wherein the illuminating device, the camera and the electronic circuitry are parts of at least one portable device.

A nineteenth preferred embodiment comprises a device in accordance with one of embodiments 13 to 18, wherein the camera and the electronic circuitry are parts of an integrated portable device.

A twentieth preferred embodiment comprises a device in accordance with one of embodiments 13 to 19, wherein the integrated portable device comprises a smartphone, a tablet or an iPad.

A twenty-first preferred embodiment comprises the device in accordance with one of embodiments 13 to 20, wherein the data exchange device comprises a data exchange device for contactless data exchange.

A twenty-second preferred embodiment comprises a device for determining a degree of colonization of skin with acne bacteria, comprising:
an illuminating device for illuminating at least one region of a user's skin with ultraviolet and/or blue light;
a camera to record an image of the region of skin during the illumination of the region of skin by the illuminating device, wherein the camera is configured to at least capture light in a fluorescence wavelength range of a fluorescent agent produced by the acne bacteria and
electronic circuitry which is configured to determine, by the image, a captured quantity of light which is emitted from the fluorescent agent as fluorescence as a consequence of the illumination, to determine a dimension for the region of skin, and to allocate a degree of colonization of the skin to the determined captured quantity of light by a database taking the dimension of the region of skin into consideration.

A twenty-third preferred embodiment comprises a device in accordance with embodiment 22, further comprising:
a distance measuring device for measuring a distance between the distance measuring device and the region of skin, wherein the determination of the dimension of the region of skin is based on the distance measuring device-region of skin distance, a camera-distance measuring device distance and camera properties.

A twenty-fourth preferred embodiment comprises a method for determining a degree of colonization of skin with acne bacteria, comprising:
for at least one region of a user's skin, recording an image of the region of skin by a camera during illumination of the region of skin with ultraviolet and/or blue light, wherein the camera is configured to at least capture light in a fluorescence wavelength range of a fluorescent agent produced by the acne bacteria;
in the image, determining a captured quantity of light which is emitted from the fluorescent agent as fluorescence as a consequence of the illumination, and determining a dimension of the region of skin; and
by a database, allocating a degree of colonization of the skin to the determined captured quantity of light taking the dimension of the region of skin into account.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A method for determining a degree of colonization of skin with acne bacteria, comprising the steps of:
for at least one region of a user's skin, during illumination of the at least one region of skin with ultraviolet and/or blue light, recording an image of the at least one region of skin using a camera, wherein the camera is configured to at least capture light in a fluorescence wavelength range of a fluorescent agent produced by the acne bacteria;
in the image, determining a captured quantity of light emitted from the fluorescent agent as fluorescence as a consequence of the illumination; and
allocating a degree of colonization of the skin to the determined captured quantity of light;
wherein the allocation of the degree of colonization of the skin to the determined captured quantity of light is carried out by a database stored in a cloud computing architecture.

2. The method as claimed in claim 1, further comprising the step of:
updating the database based on new allocations of the degree of colonization of the skin to the determined captured quantity of light from a plurality of other users.

3. The method as claimed in claim 1, further comprising the step of:
recording a background light image of the region of skin using the camera while the region of skin is not illuminated with the ultraviolet and/or blue light,
wherein the determination of the captured quantity of light emitted from the fluorescent agent as fluorescence as a consequence of the illumination; comprises a determination of a difference between the image of the at least one region of skin and the background light image.

4. The method as claimed in claim 1, wherein the fluorescent agent comprises a porphyrin.

5. The method as claimed in claim 1, wherein the fluorescence wavelength range is from about 590 nm to about 760 nm.

6. The method as claimed in claim 1, wherein the at least one region of skin comprises a plurality of regions of the user's skin.

7. The method as claimed in claim 1, wherein the camera and an illuminating device for producing the ultraviolet and/or blue light are parts of at least one portable device.

8. The method as claimed in claim 7, wherein the portable device comprises a smartphone, a tablet, or an iPad or the portable device is capable of being attached to a smartphone, a tablet or an iPad.

9. The method as claimed in claim 1, wherein the determination of the captured quantity of light and/or the allocation of the degree of colonization of the skin to the determined captured quantity of light is carried out by an app.

10. A method for determining a cosmetic skin treatment recommendation, comprising the steps of:
determining a skin condition in accordance with claim 1; and
determining a cosmetic product and/or a care tip based on the determined degree of colonization of the skin and a further database stored in the cloud computing architecture which comprises a plurality of degrees of colonization and a plurality of associated cosmetic products and/or care tips, wherein each skin condition of the plurality of skin conditions is associated with at least one suitable cosmetic product and/or at least one care tip.

11. The method as claimed in claim 10, wherein the cosmetic product and/or the care tip is suitable for the degree of colonization of the skin when, based on empirical values stored in the further database from a plurality of other users of the cosmetic product, a reduction in the degree of colonization of the skin is to be expected.

12. The method as claimed in claim 10, wherein the determination of the degree of colonization of the skin and/or the determination of the cosmetic skin treatment recommendation comprises transmitting the image and/or the degree of colonization of the skin to an external data processing device and receiving the degree of colonization of the skin and/or the cosmetic skin treatment recommendation.

13. A device for determining a degree of colonization of skin with acne bacteria, comprising:
an illuminating device for illuminating at least one region of a user's skin with ultraviolet and/or blue light;
a camera to record an image of the region of skin during the illumination of the region of skin by the illuminating device, wherein the camera is configured to at least capture light in a fluorescence wavelength range of a fluorescent agent produced by the acne bacteria;
electronic circuitry configured to determine, in the image, a captured quantity of light emitted from the fluorescent agent as fluorescence as a consequence of the illumination; and to allocate a degree of colonization of the skin to the determined captured quantity of light by a database; and
a data exchange device for exchanging data between the electronic circuitry and a cloud computing architecture, wherein by the data exchange device, the database stored in the cloud computing architecture is made available for the allocation of the degree of colonization to the quantity of light.

14. The device as claimed in claim 13 wherein the electronic circuitry is further configured to determine a dimension for the at least one region of skin and to allocate the degree of colonization of the skin to the determined captured quantity of light by the database taking the dimension of the at least one region of skin into account.

15. The method as claimed in claim 1, further comprising the steps of:
determining a dimension of the at least one region of skin; and
by the database, allocating the degree of colonization of the skin to the determined captured quantity of light taking the dimension of the at least one region of skin into account.

16. The method as claimed in claim 4 wherein the fluorescent agent comprises coproporphyrin III.
